(19) United States
(12) Patent Application Publication
(10) Pub. No.: US 2011/0124586 A1
(43) Pub. Date: May 26, 2011

(54) EXTERNAL PREPARATION FOR SKIN, AND WRINKLE-REPAIRING AGENT

(75) Inventors: Kyoko Miura, Kanagawa (JP); Akinori Haratake, Kanagawa (JP)

(73) Assignee: Kao Corporation, Tokyo (JP)

(21) Appl. No.: 13/055,651

(22) PCT Filed: Jul. 24, 2009
(86) PCT No.: PCT/JP2009/003489

§ 371 Date: Jan. 24, 2011

(30) Foreign Application Priority Data
Jul. 28, 2008 (JP) 2008-194068

(51) Int. Cl. A61K 8/60; C07H 15/14; A61Q 19/08

(52) U.S. Cl. 514/24; 536/4.1

(57) ABSTRACT

Provided are an external preparation for skin and a wrinkle-reducing agent exhibiting an excellent effect of reducing wrinkles that develop particularly in an exposed site along with aging, and having high safety. The external preparation for skin and the wrinkle-reducing agent each contain an alkyl thioglycoside represented by the general formula (1): G-SR₁ (1) (in the formula: G represents a sugar; R₁ represents an alkyl group having 10 to 18 carbon atoms; and G and an SR₁ group are bonded to each other via a β-glycosidic bond).

### FIELD OF THE INVENTION

The present invention relates to an external preparation for skin and a wrinkle-reducing agent.

### BACKGROUND OF THE INVENTION

Organs of all living organisms including humans grow from birth, then gradually lose their functions with age, and become dysfunctional at some time. When the proportion of the organs which have become dysfunctional exceeds a certain extent, the living organisms die. The process in which the functions become gradually lost is called aging. Skin is directly affected by surroundings and has important functions of maintaining the internal condition of a living body. It is therefore rare that the skin becomes completely dysfunctional. However, the skin is an organ on which aging signs such as wrinkles, liver spots, dullness, and sags are liable to appear, and those signs are particularly remarkable in a site that is exposed to sun light.

The progression of skin aging weakens an ability to defend against irritation such as oxidation stress and causes the disturbance of the skin internal condition to further progress aging. In general, histological changes in the skin with age differ in an exposed site and an unexposed site from each other and are classified into photoaging and physiological aging (Non Patent Document 1). In particular, the exposed site is always exposed to strong oxidation stress such as ultraviolet radiation and hence the progression of aging is remarkable. Photoaged skin leads to cosmetically undesirable conditions such as thickened epidermis and deep and large wrinkles. On the other hand, in physiological aging, it has been pointed out that so-called fine wrinkles appear and the moist condition of the horny layer has a high correlation with the development of such fine wrinkles (Non Patent Document 1), and that the horny layer becomes thick whereas the epidermis becomes thin (Non Patent Documents 1 and 2).

It is considered that epidermal thickening and wrinkles caused by photoaging cannot be sufficiently prevented and reduced by merely protecting skin from drying using a moisture-retaining agent or the like. In the United States, retinoic acid, which serves as a substance having an effect of reducing wrinkles resulting from the progression of photoaging, has been used as a prescription drug. However, retinoic acid has not been approved in Japan yet because of its strong adverse effects and safety concerns (Non Patent Document 3). Thus, there is a demand for a wrinkle-reducing substance which is high in safety and has a sufficient effect.

Meanwhile, it is known that an alkyl thioglycoside may be applied as a micelle surfactant into, for example, oral pharmaceuticals such as an oral agent for the treatment of diabetes (Patent Document 1) and may be employed as a membrane protein solubilizer (Patent Documents 2 and 3), and n-octyl-β-D-thio-glucopyranoside may be employed as a transdermal absorption promoter of a drug (Patent Document 4). However, no studies have been made on actions in the case of the application to skin, in particular, actions on wrinkles.

### PRIOR ART DOCUMENT

### Patent Document

[Patent Document 1] JP-A-2002-69000
[Patent Document 2] JP-A-61-7288
[Patent Document 3] JP-A-05-32689
[Patent Document 4] JP-A-63-218630

### Non Patent Document

[Non Patent Document 1] Kiichiro Danno, “Photoaging and wrinkles”, “Fragrance Journal”, Fragrance Journal Ltd., issued on Apr. 15, 1998, 26(4), pp. 11-17
[Non Patent Document 2] Setsuko Jitsukawa, Kazushige Hara, “A new xylose-derived active cosmetic ingredient for anti-aging—Toward sustainable development”, “Fragrance Journal”, Fragrance Journal Ltd., issued on Oct. 15, 2006, 34(10), pp. 35-39
[Non Patent Document 3] Yoshio Hamada, Gen Fukuse, “Retinoids as anti-wrinkle treatment”, “Fragrance Journal”, Fragrance Journal Ltd., issued on Apr. 15, 1998, 26(4), pp. 75-77

### SUMMARY OF THE INVENTION

### Problems to be Solved by the Invention

The present invention relates to an external preparation for skin and a wrinkle-reducing agent exhibiting an excellent effect of reducing wrinkles that are remarkably evident particularly in an exposed site along with aging and exhibiting an excellent effect of keeping cosmetically healthy skin.

### Means for Solving Problems

In view of the above-mentioned circumstances, the inventors of the present invention have made extensive studies. As a result, the inventors have confirmed that an alkyl thioglycoside, when being externally applied to skin, exhibits effects of reducing skin wrinkles, in particular, wrinkles that remarkably appear in an exposed site and keeping cosmetically healthy skin, and has excellent safety. Thus, the present invention has been completed.

In other words, the present invention provides an external preparation for skin, including an alkyl thioglycoside represented by the general formula (1): G-SR₁ (1) (in the formula: G represents a sugar; R₁ represents an alkyl group having 10 to 18 carbon atoms; and G and an SR₁ group are bonded to each other via a β-glycosidic bond).

The present invention also provides a wrinkle-reducing agent for reducing wrinkles due to photoaging, including an alkyl thioglycoside represented by the general formula (2): G-SR₂ (2) (in the formula: G represents a sugar; R₂ represents an alkyl group having 8 to 18 carbon atoms; and G and an SR₂ group are bonded to each other via a β-glycosidic bond).

The present invention also provides use for the production of a wrinkle-reducing agent for wrinkles due to photoaging of an alkyl thioglycoside (2) represented by the general formula (2): G-SR₂ (2) (in the formula: G represents a sugar; R₂ represents an alkyl group having 8 to 18 carbon atom; and G and an SR₂ group are bonded to each other via a β-glycosidic bond).

The present invention also provides a method of reducing wrinkles due to photoaging of skin, including applying to the skin an alkyl thioglycoside represented by the general formula (2): G-SR₂ (2) (in the formula: G represents a sugar; R₂ represents an alkyl group having 8 to 18 carbon atoms; and G and an SR₂ group are bonded to each other via a β-glycosidic bond).

### EFFECTS OF THE INVENTION

The present invention can provide an external preparation for skin and a wrinkle-reducing agent exhibiting excellent effects of reducing wrinkles markedly formed in an exposed site along with aging and keeping the skin dermatologically and cosmetically healthy.

### BRIEF DESCRIPTION OF DRAWING

FIG. 1 is a graph illustrating a rate of decrease in water content (average value for n=3) of lauryl thiogalactoside solution.

### DETAILED DESCRIPTION OF THE INVENTION

An alkyl thioglycoside to be used in the present invention is represented by the general formula (1) or (2). A sugar residue represented by G is, for example, a monosaccharide, a disaccharide, or an oligosaccharide, preferably, for example, glucose, galactose, lactose, or maltose, more preferably glucose or galactose.

Further, an alkyl chain having 10 to 18 carbon atoms, preferably 10 to 12 carbon atoms may be used as an alkyl chain represented by R₁ serving as an aglycone moiety. Moreover, an alkyl chain having 8 to 18 carbon atoms, preferably 8 to 12 carbon atoms may be used as an alkyl chain represented by R₂. Specific examples of the alkyl chain include octyl (C8), ethylhexyl (C8), decyl (C10), lauryl (C12), myristyl (C14), palmityl (C16), stearyl (C18), isostearyl (C18), and oleyl (C18). The above-mentioned range is preferred because an external preparation for skin, to which the alkyl chain is applied, is excellent in terms of solubility and the like, and is also excellent in terms of ease of synthesis and physical properties.

In addition, any of linear and branched and saturated and unsaturated alkyl chains may be used as the alkyl chain. Of those, a saturated and linear chain is preferred.

More preferred examples of the alkyl thioglycoside include a C₈ to C₁₂ alkyl thioglucoside and a C₈ to C₁₂ alkyl thiogalactoside. To be specific, octyl thioglucoside, decyl thioglucoside, lauryl thioglucoside, octyl thiogalactoside, decyl thiogalactoside, and lauryl thiogalactoside are more preferred.

In addition, lauryl thioglucoside represented by the following formula (3), lauryl thiogalactoside represented by the formula (4), and octyl thioglucoside represented by the formula (5) are even more preferred.

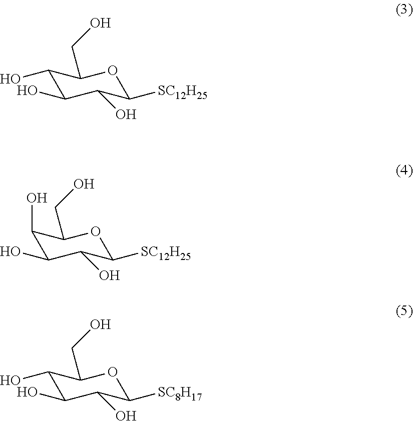

When the alkyl thioglycoside represented by the above-mentioned general formula (1) or (2) is used as a wrinkle-reducing agent for reducing wrinkles due to photoaging, the alkyl group represented by R₂ in the general formula (2) has 8 to 18 carbon atoms. On the other hand, when the alkyl thioglycoside is used as an external preparation for skin, the alkyl group represented by R₁ in the general formula (1) has 10 to 18 carbon atoms.

A synthesis method for the alkyl thioglycoside represented by the general formula (1) or (2) is, for example, a method described below.

For example, in a synthesis method for the above-mentioned structural formula (4), the alkyl thioglycoside may be produced by allowing commercially available 1,2,3,4,6-penta-O-acetyl-D-galactopyranose as a starting material to react with 1-dodecanethiol and boron trifluoride etherate (BF₃.OEt₂) in a chloroform solvent and then subjecting the resultant to a deacetylation treatment.

In the above-mentioned synthesis method, a variety of alkyl thioglycosides represented by the general formula (1) or (2) may be obtained by using, for example, 1,2,3,4,6-penta-O-acetyl-D-glucopyranose in place of 1,2,3,4,6-penta-O-acetyl-D-galactopyranose, and using, for example, commercially available 1-octanethiol, 1-nonanethiol, 1-decanethiol, 1-undecanethiol, 1-tetradecanethiol, 1-pentadecanethiol, 1-hexadecanethiol, or 1-octadecanethiol in place of 1-dodecanethiol.

Further, a commercially available alkyl thioglycoside may be purchased and used. For example, decyl 1-thio-β-D-glucopyranoside and octyl 1-thio-β-D-glucopyranoside (manufactured by SIGMA-ALDRICH) may be used.

When the alkyl thioglycoside represented by the general formula (1) or (2) is applied to skin as described in test examples and examples described later, the alkyl thioglycoside has actions of suppressing the progression of skin wrinkles, in particular, wrinkles due to photoaging, and reducing wrinkle symptoms. Accordingly, the application of a composition containing the alkyl thioglycoside to the skin can reduce wrinkles. Further, the composition containing the alkyl thioglycoside is useful as an external preparation for skin for reducing wrinkles.

The content of the alkyl thioglycoside (1) or (2) in the external preparation for skin or the wrinkle-reducing agent of the present invention is preferably 0.001 to 10 mass % (hereinafter, simply referred to as %), more preferably 0.01 to 5% on the basis of the total amount of the external preparation for skin or the wrinkle-reducing agent. A content within the above-mentioned range provides a sufficient wrinkle-reducing effect.

The form of the external preparation for skin or the wrinkle-reducing agent of the present invention may be any form as long as the form is applicable to skin. Examples of the form include solutions, milky lotions, creams, lotions, gels, packs, and bath agents.

It should be noted that the external preparation for skin and the wrinkle-reducing agent of the present invention may appropriately contain, in addition to the above-mentioned components, for example, dyes, perfumes, preservatives, surfactants, pigments, anti-oxidants, skin-whitening agents, moisture-retaining agents, ultraviolet absorbing agents, ultraviolet scattering agents, oily components, thickeners, alcohols, organic acids, pH adjustors, or water in such a range that the object of the present invention is achieved.

### EXAMPLES

Hereinafter, the present invention is described in detail based on production examples, examples, and comparative examples.

### Production Example 1

### Method of Producing Lauryl Thioglucoside Represented by Formula (3)

To a solution obtained by dissolving 1,2,3,4,6-penta-O-acetyl-D-glucopyranose (1 g) in chloroform (10 mL) were added 1-dodecanethiol (0.57 g) and BF₃.OEt₂ (1.6 mL), and the mixture was stirred. After the completion of the reaction, the reaction solution was washed with a sodium hydrogen carbonate aqueous solution, and then, a chloroform layer was dried over anhydrous magnesium sulfate. Subsequently, a solvent was distilled off under reduced pressure. The resultant residue was subjected to a deacetylation reaction using sodium methylate in a methanol solvent to afford lauryl thioglucoside (0.68 g, total yield: 72%) of interest.

### Production Example 2

### Method of Producing Lauryl Thiogalactoside Represented by Formula (4)

To a solution obtained by dissolving 1,2,3,4,6-penta-O-acetyl-β-D-galactopyranose (1 g) in chloroform (10 mL) were added 1-dodecanethiol (0.57 g) and BF₃.OEt₂ (1.6 mL), and the mixture was stirred. After the completion of the reaction, the reaction solution was washed with a sodium hydrogen carbonate aqueous solution, and then, a chloroform layer was dried over anhydrous magnesium sulfate. Subsequently, a solvent was distilled off under reduced pressure. The resultant residue was subjected to a deacetylation reaction using sodium methylate in a methanol solvent to afford lauryl thiogalactoside (0.56 g, total yield: 60%) of interest.

Moisture Retention Test Example

In accordance with the following test method, the rate of decrease in water content of lauryl thiogalactoside (compound of Production Example 2) solution was calculated to evaluate moisture-retaining property.

(1) Test Methods

0.5 g of a sample prepared by adjusting a concentration of lauryl thiogalactoside to 10 mass % with ion exchanged water was weighed in a container made of a resin to measure an initial weight (W0).

Next, the sample was left to stand still in a silica gel desiccator (RT; 25° C., RH; 20%) to measure a time-dependent weight (Wt) of the sample.

After the completion of the test, the sample was dried in a dryer (50° C.) for 2 hours and then left to stand still in a silica gel desiccator for 2 hours to measure a dry weight (Ws).

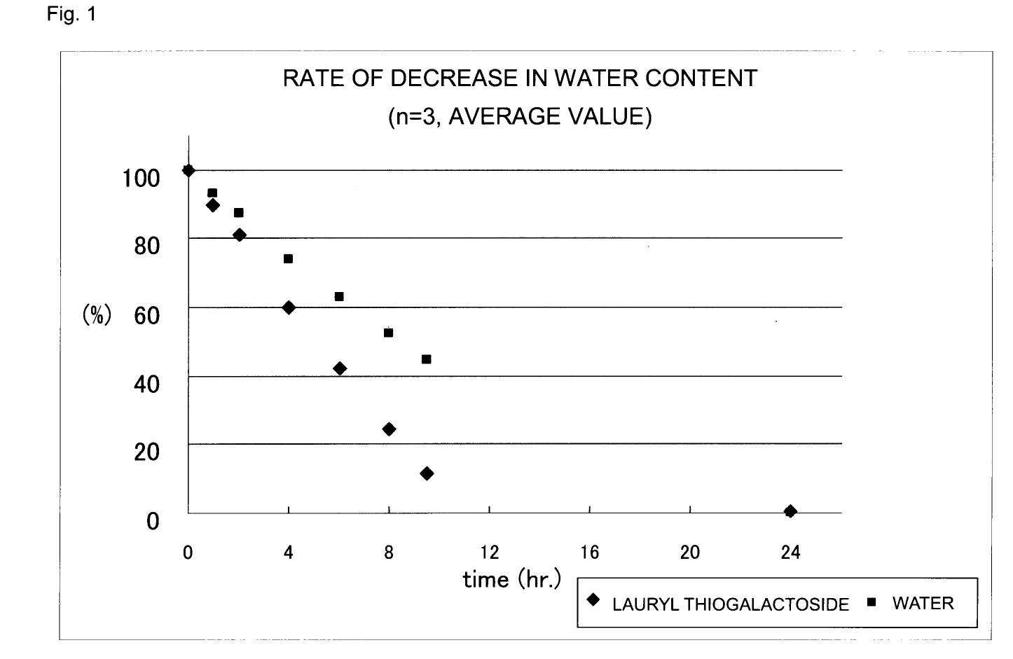

The same operation was conducted for a control sample prepared using water alone, and the rate of decrease in water content was calculated with the following equation. FIG. 1 shows the results.

(2) Calculation Equation

Rate of decrease in water content (%)=Ht/H0×100(Ht=Wt−Ws, H0=W0−Ws)

FIG. 1 revealed that lauryl thiogalactoside did not suppress water volatilization in a dry state and exhibited an insufficient moisture-retaining action.

Wrinkle Reduction Test Example

A wrinkle-reducing effect in the case of applying a sample containing a base alone or lauryl thiogalactoside (compound of Production Example 2) to photoaged skin was examined by the following test methods.

(1) Experimental Animals

Ten hairless mice, which were 10 weeks old at the start of the test, were used for each group.

(2) Measurement of Wrinkle-Reducing Effect

### (2)-1. Photoaging Conditions and Measurement Methods

Photoaging was induced by irradiation with UVA and UVB once a day, five times a week for 8 weeks. An irradiation dose was increased every week from 20 J/cm² to 25 J/cm² and to 30 J/cm² for UVA, from 20 mJ/cm² to 30 mJ/cm² and to 40 mJ/cm² for UVB, and the maximum dose was irradiated in Week 3 or later.

A wrinkle-reducing effect was evaluated by a wrinkle score and an epidermal thickness. The wrinkle score was determined in accordance with the method of Bissett et al. (Photochem. Photobiol. 46: 367-378, 1987). In other words, the size and depth of wrinkles were comprehensively evaluated with naked eyes and scored with the following four grades: 3, “large and deep wrinkles can be confirmed”; 2, “wrinkles can be confirmed”; 1, “no wrinkles can be confirmed”; and 0, “normal skin texture can be observed”. The measurement of the epidermal thickness was performed by collecting the whole layer skin, and preparing a skin section in accordance with a conventional method, then subjecting the skin section to hematoxylin-eosin staining, and measuring the epidermal thickness with image analysis software (Microanalyzer manufactured by Nihon Poladigital, K. K.).

(2)-2. Samples and Experimental Methods

A sample containing lauryl thiogalactoside in an amount of 1% in a 50 vol % ethanol aqueous solution (base) was prepared (Example 1). Further, a sample containing a base alone was prepared as Comparative Example 1. First, 0.1 mL of each of those samples was applied onto the dorsal skin (about 2.5 cm in diameter) of hairless mice with a frequency of once a day, five times a week, from Week 5 after the start of UV irradiation to Week 4 after the completion of the irradiation. Subsequently, after the completion of the application, the wrinkle score was determined. The mice had been sacrificed, and the skin was then collected. Both of the wrinkle score and the epidermal thickness were compared to those of a base-applied group as a control. Tables 1 and 2 show the results.

TABLE 1
(Wrinkle score evaluation results)

| | Group | Wrinkle score |
|---|---|---|
| Example 1 | Lauryl | 2.4 ± 0.2** |
| | thiogalactoside-containing | (p = 0.0071) |
| | sample-applied group | |
| Comparative | Base sample-applied group | 2.8 ± 0.1 |
| Example 1 | | |

(The values are each calculated as an average value ± a standard error, and the statistically significant difference was verified by a Dunnett's multiple comparison test.)

Table 1 revealed that Example 1 showed a significantly low wrinkle score value as compared to Comparative Example 1, and lauryl thiogalactoside was thus effective for wrinkles induced by photoaging.

TABLE 2
(Epidermal thickness measurement results)

| | | Epidermal |
|---|---|---|
| | Group | thickness (μm) |
| Example 1 | Lauryl | 25.94 ± 1.61** |
| | thiogalactoside-containing | (p = 0.0062) |
| | sample-applied group | |
| Comparative | Base sample-applied group | 35.38 ± 2.77 |
| Example 1 | | |

(The values were each calculated as an average value ± a standard error, and the statistically significant difference was verified by a Dunnett's multiple comparison test.)

Table 2 demonstrated that the wrinkle-reducing agent-applied group of Example 1 showed a significantly thin epidermal thickness as compared to that of the applied group of Comparative Example 1, and lauryl thiogalactoside had an effect of alleviating the epidermal thickening owing to photoaging. It should be noted that, when retinoic acid is applied in this test system, retinoic acid is effective for the wrinkle score but has a function of enhancing the thickening with regard to the epidermal thickness. This action has been one factor of safety concerns. In contrast, lauryl thiogalactoside does not have any such action and also does not have any problem in an ordinary safety test.

The test results reveal that the wrinkle-reducing agent containing lauryl thiogalactoside of Example 1 clearly has an effect of reducing wrinkles due to photoaging as compared to that of Comparative Example 1 and the effect is not based on the moisture-retaining action of lauryl thiogalactoside.

### Example 2

In this example and comparative example, skin creams having the following compositions were prepared in accordance with a preparation method described below. The skin creams were used as samples and evaluated for their wrinkle-reducing effects in accordance with the following procedure.

To five healthy volunteers (females, 43 to 56 years old) who, in questionnaires before the test, mentioned wrinkles at the outer corners of the eyes as a skin problem, the skin cream of Example 2 or Comparative Example 2 was applied. Then, a questionnaire survey on the condition of the skin (wrinkles) at the outer corners of the eyes was carried out in accordance with the following method. Each sample was applied onto the wrinkle portion of any one of the right or left outer corners of the eyes (about 4 cm², 2×2 cm around the outer corner of the eye for each sample) in an unit dose of about 0.2 mL twice a day, after face washing in the morning and after bathing in the evening for two consecutive months (60 days). Next, the volunteers answered questionnaires on the conditions of the skin (wrinkles) at the right and left outer corners of the eyes after the completion of the final application.

TABLE 3
Composition of skin cream

| Raw material component | Blending amount (%) |
|---|---|
| Component A | |
| Beeswax | 2.0 |
| Stearic acid | 5.0 |
| Stearyl alcohol | 5.0 |
| Hydrogenated lanolin | 2.0 |
| Squalene | 20.0 |
| Sorbitan monostearate | 3.0 |
| Polyoxyethylene (20) sorbitan | 3.0 |
| monostearate | |
| Propylene glycol | 5.0 |
| Component B | |
| Methylparaben | 0.2 |
| Purified water | Balance |
| Component C | |
| Lauryl thioglucoside (compound of | 1.0 (Example 2) |
| Production Example 1) | or 0 (Comparative Example 2) |
| Total | 100 |

Preparation Method

Each skin cream was prepared by adding lauryl thioglucoside as the component C to the component B, dissolving each of the components A and B by heating to 80° C., and then cooling the resultant to 30° C. while mixing and stirring.

Based on the questionnaire results, in each of items concerning the conditions of skin (wrinkles), the number of persons who answered that the skin cream of Example 2 was more effective than that of Comparative Example 2 are shown below.

TABLE 4

| Item | Number of persons (persons) |
|---|---|
| Wrinkles became less noticeable | 5 |
| Skin became soft | 3 |
| Skin became elastic | 4 |
| Skin became shiny | 4 |
| Skin become bright | 4 |

The test results reveal that the skin cream of Example 2 clearly reduced wrinkles and further improved suppleness and elasticity of skin, which were deteriorated by photoaging, as compared to that of Comparative Example 2. Further, no skin abnormality such as irritation or itching due to the skin cream of the present invention was observed.

### Example 3

A skin lotion having the following composition was prepared in accordance with a conventional method. The skin lotion was used for 1 month by 20 healthy volunteers (female, 43 to 56 years old) who, in questionnaires before the test, mentioned wrinkles at the outer corners of the eyes as a skin problem, to perform a questionnaire survey.

TABLE 5
Composition of skin lotion

| Raw material component | Blending amount (%) |
|---|---|
| Ethanol | 8.0 |
| POE (60) hydrogenated castor oil | 0.3 |
| Polyoxyethylene (20) sorbitan | 0.1 |
| monolaurate | |
| Glycerin | 1.0 |
| Polyethylene glycol 4000 | 0.1 |
| Disodium phosphate | 0.09 |
| Monopotassium phosphate | 0.03 |
| Disodium edetate | 0.02 |
| Methylparaben | 0.1 |
| Octyl thioglucoside (*1) | 1.0 |
| Purified water | Balance |
| Total | 100 |

*1; Octyl 1-thio-β-D-glucopyranoside manufactured by SIGMA-ALDRICH

The skin lotion of Example 3 was used by the volunteers to perform a questionnaire survey. The results are shown below. It should be noted that the results show the number of persons who answered “yes” with respect to each item, comparing the conditions before use and after use, in a questionnaire survey performed on the following items concerning the conditions of wrinkles.

TABLE 6

| Item | Number of persons (number) |
|---|---|
| Wrinkles became less noticeable | 16 |
| The size of wrinkles decreased | 15 |
| The number of wrinkles decreased | 7 |
| Wrinkles increased | 0 |

The test results reveal that almost all the persons feel that the skin lotion of Example 3 made wrinkles less noticeable as compared to the condition before use, which was based on the reduction of wrinkles due to photoaging through a decrease in the size of wrinkles rather than a decrease in the number of wrinkles. Further, no skin abnormality such as irritation or itching due to the skin lotion of the present invention was observed.

### Examples 4 and 5

### Milky Lotion

The milky lotion of the present invention was prepared by a conventional method in accordance with the following composition.

TABLE 7

| | Blending amount (%) | |
|---|---|---|
| Raw material component | Example 4 | Example 5 |
| Hydrogenated lecithin | 1.0 | 1.0 |
| Cholesterol | 0.5 | 0.5 |
| Squalane | 5.0 | 5.0 |
| Octyldodecyl myristate | 3.0 | 3.0 |
| Dipropylene glycol | 4.0 | 4.0 |
| 1,3-Butylene glycol | 4.0 | 4.0 |
| Glycerin | 7.0 | 7.0 |
| Diglycerin | 2.0 | 2.0 |
| Phenoxyethanol | 0.1 | 0.1 |
| Disodium edetate | 0.02 | 0.02 |
| Potassium hydroxide | q.s. | q.s. |
| Xanthan gum | 0.1 | 0.1 |
| Alkyl acrylate/methacrylate | 0.08 | 0.08 |
| copolymer (*2) | | |
| Carboxyvinyl polymer (*3) | 0.3 | 0.3 |
| Lauryl thioglucoside | 1.0 | — |
| Stearyl thioglucoside | — | 0.5 |
| Perfume | 0.01 | 0.01 |
| Purified water | Balance | Balance |

| | |
|---|---|
| Total | 100 |

*2; PEMULEN TR-1 manufactured by Lubrizol Advanced Materials
*3; Synthalene L manufactured by 3V SIGMA

The milky lotion showed satisfactory results in the above-mentioned test.

### Examples 6 and 7

### Day Essence Cosmetic

The day essence cosmetic of the present invention was prepared by a conventional method in accordance with the following composition.

TABLE 8

| | Blending amount (%) | |
|---|---|---|
| Raw material component | Example 6 | Example 7 |
| Ethanol | 10.0 | 10.0 |
| Phenoxyethanol | 0.3 | 0.3 |
| Polyoxyethylene (20) sorbitan | 0.4 | 0.4 |
| monolaurate | | |
| POE (60) hydrogenated castor oil | 0.8 | 0.8 |
| Methylpolysiloxane (*4) | 2.0 | 2.0 |
| Methylphenylpolysiloxane (*5) | 0.5 | 0.5 |
| Squalane | 0.5 | 0.5 |
| Disodium edetate | 0.02 | 0.02 |
| Polyethylene glycol 4000 | 6.0 | 6.0 |
| Glycerin | 10.0 | 10.0 |
| Dipropylene glycol | 4.0 | 4.0 |
| Xanthan gum | 0.04 | 0.04 |
| Carboxyvinyl polymer (*6) | 0.3 | 0.3 |
| Potassium hydroxide | q.s. | q.s. |
| Lauryl thioglucoside | 1.0 | — |
| Lauryl thiogalactoside | — | 1.0 |
| Perfume | 0.05 | 0.05 |
| Purified water | Balance | Balance |

| | |
|---|---|
| Total | 100 |

*4; Silicone KF-96A (100CS) manufactured by Shin-Etsu Chemical Co., Ltd.
*5; Silicone FZ-209 manufactured by Dow Corning Toray Co., Ltd.
*6; Carbopol 940 manufactured by Lubrizol Advanced Materials

The day essence cosmetic showed satisfactory results in the above-mentioned test.

### Examples 8 and 9

### Sunscreen

The sunscreen of the present invention was prepared by a conventional method in accordance with the following composition.

TABLE 9

| | Blending amount (%) | |
|---|---|---|
| Raw material component | Example 8 | Example 9 |
| Ethanol | 10.0 | 10.0 |
| Octyl methoxycinnamate | 7.0 | 7.0 |
| Poly (oxyethylene/oxypropylene) me | 2.0 | 2.0 |
| thylpolysiloxane copolymer (*7) | | |
| Fine titanium oxide particles | 5.0 | 5.0 |
| Zinc oxide | 5.0 | 5.0 |
| Methylcyclopolysiloxane (*8) | 10.0 | 10.0 |
| Egg yolk lecithin | 2.0 | 2.0 |
| Lauryl thioglucoside | 1.0 | — |
| Stearyl thiogalactoside | — | 0.5 |
| Perfume | 0.1 | 0.1 |
| Purified water | Balance | Balance |

| | |
|---|---|
| Total | 100 |

*7; Silicone BY22-008 manufactured by Dow Corning Toray Silicone Co., Ltd.
*8; TSF405 manufactured by Momentive Performance Materials Inc. The sunscreen showed satisfactory results in the above-mentioned test.

It should be noted that a perfume having the following perfume prescription was used as the perfume in Examples.

TABLE 10
Perfume prescription A

| Component | mass % |
|---|---|
| Terpineol | 10.00 |
| Terpinyl acetate | 2.00 |
| Cepionate | 60.00 |
| Methyl dihydrojasmonate | 250.00 |
| Indole | 0.05 |
| 2-Methyl-3-(3,4-methylene- | 3.00 |
| dioxy-phenyl)-propanal | |
| Hydroxycitronellal | 20.00 |
| Hydroxycitronellol | 10.00 |
| p-t-Butyl-α-methylhydro- | 35.00 |
| cinnamic-aldehyde | |
| 4-(4-Hydroxy-4-methyl- | 75.00 |
| pentyl)-3-cyclohexene- | |
| 1- carboxyaldehyde | |
| 3-Methyl-5-phenylpentanol | 20.00 |
| Phenylethyl alcohol | 10.00 |
| α-Ionone | 10.00 |
| β-Ionone | 20.00 |
| γ-Methyl ionone | 10.00 |
| Dihydro-β-ionone | 25.00 |
| Benzyl salicylate | 150.00 |
| cis-3-Hexenyl salicylate | 30.00 |
| Eugenol | 0.80 |
| Cinnamic alcohol | 5.00 |
| Cinnamic aldehyde | 0.50 |
| Guaiol acetate | 1.00 |
| Guaiol | 0.50 |
| Cedrenyl acetate | 5.00 |
| Cedryl methyl ketone | 30.00 |
| 6,7-Dihydro-1,1,2,3,3- | 2.00 |
| pentamethyl-4 (5H)-indan | |
| Vetiver acetate | 10.00 |
| 3-Methyl-5-(2,3,3- | 2.00 |
| trimethyl-3-cyclopenten-1-yl)- | |
| pentan-2-ol | |
| 2-Ethyl-4-(2,3,3-trimethyl-3- | 0.80 |
| cyclopenten-1-yl)-2- | |
| buten-1-ol | |
| Isobornyl cyclohexanol | 35.00 |
| Heliotropin | 10.00 |
| Coumarin | 2.00 |
| Vanillin | 2.00 |
| Ethyl vanillin | 0.10 |
| Muscone | 0.50 |
| Ethylene brassylate | 42.00 |
| 4,6,6,7,8,8-Hexamethyl-1,3,4, | 60.00 |
| 6,7,8-hexa-hydrocyclopenta- | |
| benzopyran | |
| Cyclopentadec-anolide | 20.00 |
| Ambrettolide | 1.00 |
| γ-Undecalactone | 0.40 |
| γ-Decalactone | 0.10 |
| 4-(4-Hydroxyphenyl)-2-butanone | 0.50 |
| Musk ketone | 0.10 |
| Skatole | 0.01 |
| cis-Jasmone | 0.05 |
| Phenylethyl acetate | 0.10 |
| Civetone | 0.20 |
| γ-Nonalactone | 0.05 |
| α-Santalol | 0.20 |
| β-Santalol | 0.20 |
| Eugenyl acetate | 0.10 |
| α-Hexyl cinnamic aldehyde | 20.00 |
| α-Damascone | 0.04 |
| β-Damascone | 0.02 |
| β-Damascenone | 0.01 |
| δ-Damascone | 0.01 |
| Rose absolute | 0.50 |
| Rose oil | 4.50 |
| Sandal wood oil | 2.00 |
| Labdanum absolute | 0.05 |
| Cistus absolute | 0.01 |
| Vetiver oil | 0.50 |
| Guaiac wood oil | 0.10 |
| Total | 1000.00 |

### INDUSTRIAL APPLICABILITY

The composition of the present invention can be applied as the external preparation for skin and the wrinkle-reducing agent to, for example, pharmaceuticals, quasi drugs, and cosmetics, can be formed into dosage forms such as lotions, milky lotions, creams, packs, and bath agents, and is thus very useful from the viewpoint of the beauty of skin.

1. An external preparation for skin, comprising an alkyl thioglycoside represented by the general formula (1):
G-SR₁ (1)
wherein G represents a sugar, R₁ represents an alkyl group having 10 to 18 carbon atoms, and G and an SR₁ group are bonded to each other via a β-glycosidic bond.

2. The external preparation for skin according to claim 1, wherein the alkyl thioglycoside is a C₁₀ to C₁₂ alkyl thioglucoside or a C₁₀ to C₁₂ alkyl thiogalactoside.

3. The external preparation for skin according to claim 1, wherein the alkyl thioglycoside is lauryl thioglucoside or lauryl thiogalactoside.

4. A wrinkle-reducing agent for reducing wrinkles due to photoaging, comprising an alkyl thioglycoside represented by the general formula (2):
G-SR₂ (2)
wherein G represents a sugar, R₂ represents an alkyl group having 8 to 18 carbon atoms, G and an SR₂ group are bonded to each other via a β-glycosidic bond.

5. The wrinkle-reducing agent according to claim 4, wherein the alkyl thioglycoside is a C₈ to C₁₂ alkyl thioglucoside or a C₈ to C₁₂ alkyl thiogalactoside.

6. The wrinkle-reducing agent according to claim 4, wherein the alkyl thioglycoside is lauryl thioglucoside, lauryl thiogalactoside, or octyl thioglucoside.

7. Use for production of a wrinkle-reducing agent for reducing wrinkles due to photoaging of an alkyl thioglycoside represented by the general formula (2):
G-SR₂ (2)
wherein G represents a sugar, R₂ represents an alkyl group having 8 to 18 carbon atoms, and G and an SR₂ group are bonded to each other via a β-glycosidic bond.

8. The use according to claim 7, wherein the alkyl thioglycoside is a C₈ to C₁₂ alkyl thioglucoside or a C₈ to C₁₂ alkyl thiogalactoside.

9. The use according to claim 7, wherein the alkyl thioglycoside is lauryl thioglucoside, lauryl thiogalactoside, or octyl thioglucoside.

10. A method of reducing wrinkles due to photoaging of skin, comprising applying to the skin an alkyl thioglycoside represented by the general formula (2):
G-SR₂ (2)
wherein G represents a sugar, R₂ represents an alkyl group having 8 to 18 carbon atoms, and G and an SR₂ group are bonded to each other via a β-glycosidic bond.

11. The method according to claim 10, wherein the alkyl thioglycoside is a C₈ to C₁₂ alkyl thioglucoside or a C₈ to C₁₂ alkyl thiogalactoside.

12. The method according to claim 10, wherein the alkyl thioglycoside is lauryl thioglucoside, lauryl thiogalactoside, or octyl thioglucoside.
